(19) United States
(12) Patent Application Publication
(10) Pub. No.: US 2006/0173714 A1
(43) Pub. Date: Aug. 3, 2006

(54) Apparatus, system, and method for facilitating compliance with guidelines for pharmaceutical preparations

(76) Inventor: Raymond P. Grotzinger JR., Dublin, OH (US)

Correspondence Address: COOLEY GODWARD, LLP, 3000 EL CAMINO REAL, 5 PALO ALTO SQUARE, PALO ALTO, CA, 94306, US

(21) Appl. No.: 11/317,906

(22) Filed: Dec. 22, 2005

Related U.S. Application Data
(60) Provisional application No. 60/638,796, filed on Dec. 22, 2004.

(51) Int. Cl. G06Q 10/00; A61B 5/00

(52) U.S. Cl. 705/2; 600/300

(57) ABSTRACT

An apparatus, a system, and a method for facilitating compliance with guidelines for pharmaceutical preparations are described. In one embodiment, a computer-readable medium includes a documentation module to track activities related to pharmaceutical compounding. The computer-readable medium also includes a reporting module to evaluate the activities so as to characterize a level of compliance with a pharmaceutical compounding guideline.

### CROSS-REFERENCE TO RELATED APPLICATION

This application claims the benefit of U.S. Provisional Application Ser. No. 60/638,796, filed on Dec. 22, 2004, the disclosure of which is incorporated herein by reference in its entirety.

### BRIEF DESCRIPTION OF THE INVENTION

The invention relates generally to pharmaceutical preparations. More particularly, the invention relates to an apparatus, a system, and a method for facilitating compliance with guidelines for pharmaceutical preparations.

### BACKGROUND OF THE INVENTION

The Joint Commission on Accreditation of Healthcare Organizations (“JCAHO”) sets quality and safety standards for healthcare organizations and awards accreditation to those organizations that sufficiently meet the quality and safety standards. Starting in 2004, the JCAHO began surveying healthcare organizations for compliance with a recently added chapter of the United States Pharmacopoeia-National Formulary (“USP-NF”). This chapter, which is entitled “Chapter 797, Pharmaceutical Compounding—Sterile Preparations,” sets forth guidelines for a particular class of pharmaceutical preparations, namely compounded sterile preparations. As can be appreciated, compounding typically refers to mixing or otherwise manipulating a set of one or more ingredients to yield a pharmaceutical product, such as a drug, and a sterile preparation typically differs from a non-sterile preparation in that the former includes a requirement for sterility.

Chapter 797 of the USP-NF (“USP-NF 797”) classifies compounded sterile preparations with respect to three risk categories, namely low, medium, and high, depending on a potential for microbial contamination. This contamination can result from, for example, use of non-sterile ingredients or equipment, complex or prolonged procedures, open exposure to an outside environment, or prolonged storage time between compounding and initiation of administration. These risk categories have varying and detailed requirements in terms of procedures and environments to be used when preparing compounded sterile products. In addition, USP-NF 797 sets forth detailed requirements in terms of evaluation and training of personnel involved in compounded sterile preparations.

Enforcement of USP-NF 797 is expected to have a wide ranging impact on healthcare organizations, including hospital pharmacies, home health care agencies, specialty clinics, and other non-manufacturing healthcare facilities that treat patients with compounded sterile products. In particular, to comply with the requirements of USP-NF 797, an affected healthcare organization should ensure not only that its operation and infrastructure meet those requirements but also that detailed information regarding its operation and infrastructure is collected so as to allow reporting to the JCAHO.

It is against this background that a need arose to develop the apparatus, the system, and the method described herein.

### SUMMARY OF THE INVENTION

In one aspect, the invention relates to a computer-readable medium. In one embodiment, the computer-readable medium includes a documentation module to track activities related to pharmaceutical compounding. The computer-readable medium also includes a reporting module to evaluate the activities so as to characterize a level of compliance with a pharmaceutical compounding guideline.

Other aspects and embodiments of the invention are also contemplated. The foregoing summary and the following detailed description are not meant to restrict the invention to any particular embodiment but are merely meant to describe some embodiments of the invention.

### BRIEF DESCRIPTION OF THE DRAWINGS

For a better understanding of the nature and objects of some embodiments of the invention, reference should be made to the following detailed description taken in conjunction with the accompanying drawings, in which:

FIG. 1 illustrates a computer system that is implemented in accordance with an embodiment of the invention; and

FIG. 2, FIG. 3, and FIG. 4 illustrate examples of user-interface screens that are provided in accordance with an embodiment of the invention.

### DETAILED DESCRIPTION

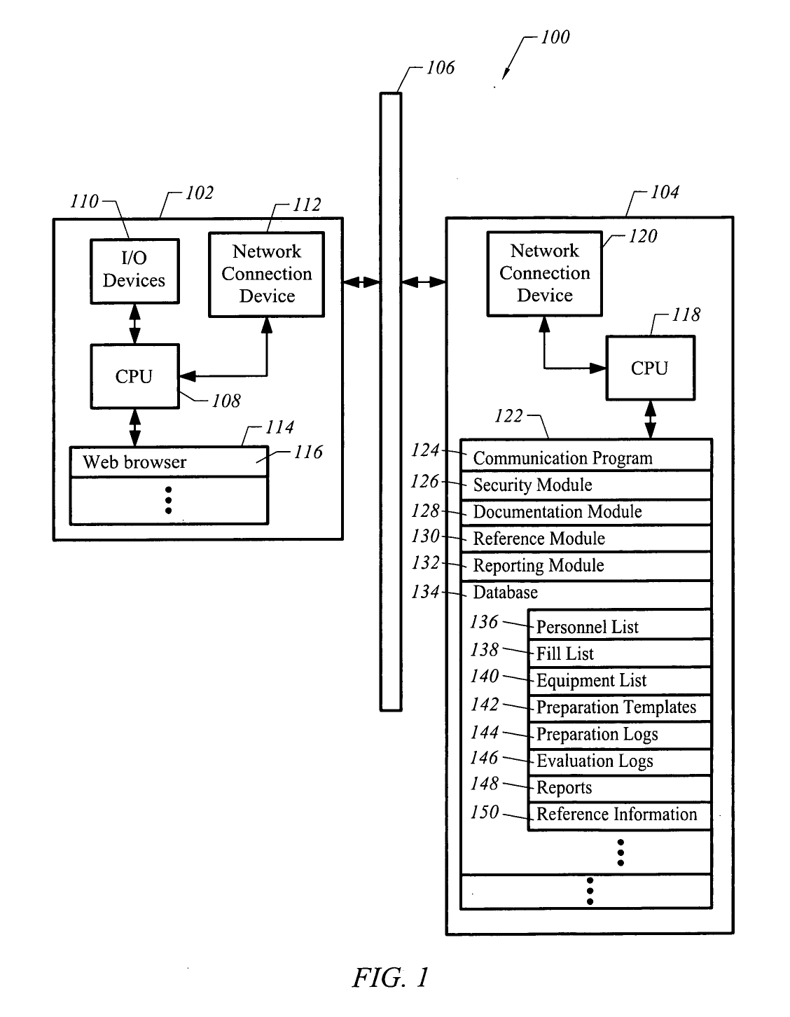

FIG. 1 illustrates a computer system 100 that is implemented in accordance with an embodiment of the invention. In the illustrated embodiment, the computer system 100 is a client-server computer network that includes at least one client computer 102 and at least one server computer 104. For example, the computer system 100 can be a Local Area Network (“LAN”), a Wide Area Network (“WAN”), or a portion of the Internet. The client computer 102 and the server computer 104 are connected by a transmission channel 106, which can be any wire or wireless transmission channel. For example, the transmission channel 106 can be a cable connection, such as one implemented in accordance with 10/100 Ethernet specifications, or a wireless connection, such as one implemented in accordance with 802.11b or 802.11g specifications.

In the illustrated embodiment, the client computer 102 is operated by a healthcare organization that prepares pharmaceutical products for treating patients. For example, the healthcare organization can be a hospital pharmacy that prepares compounded sterile products, and the client computer 102 can be operated by a user, such as a preparer, a quality assurance pharmacist, a pharmacy administrator, or any other individual who plays a role in preparing the compounded sterile products or ensuring quality and safety of the compounded sterile products.

The client computer 102 can be, for example, a desktop computer, a laptop computer, a palm-sized computer, a pen tablet computer, or a personal digital assistant. Depending on the particular implementation within the healthcare organization, the client computer 102 can be stationary or mobile, such as coupled to a cart with wheels. Referring to FIG. 1, the client computer 102 includes a number of conventional client computer components, including a Central Processing Unit (“CPU”) 108 that is connected to a set of Input/Output (“I/O”) devices 110 (e.g., a keyboard; a mouse; a stylus, such as a disposable stylus; a touch screen; a video monitor; a Radio Frequency Identification (“RFID”) reader; a biometric scanner; and a bar code reader), a network connection device 112, and a memory 114. The memory 114 stores a number of computer programs, including a web browser 116. The web browser 116 operates to establish conventional network communications with the server computer 104 via the network connection device 112. In addition, the web browser 116 operates to display information received from the server computer 104.

In the illustrated embodiment, the server computer 104 is operated as a web site to facilitate compliance with a set of guidelines for pharmaceutical preparations, such as the guidelines for compounded sterile preparations that are set forth in USP-NF 797. For example, the server computer 104 can be operated as an Intranet site by the healthcare organization or another organization that is affiliated with the healthcare organization. As another example, the server computer 104 can be operated as an Internet site by a separate organization.

Referring to FIG. 1, the server computer 104 includes a number of conventional server computer components, including a CPU 118 that is connected to a network connection device 120 and a memory 122. The memory 122 stores a number of computer programs, including a communication program 124. The communication program 124 operates to establish conventional network communications with the client computer 102 via the network connection device 120. In some instances, the communication program 124 also operates to establish conventional network communications with a pharmacy information management system (not illustrated in FIG. 1) via a conventional interface, such as one implemented in accordance with Health Level-7 (“HL-7”) interface specifications.

In the illustrated embodiment, the memory 122 also stores a set of computer programs that implement the operations described herein. In particular, the memory 122 stores a security module 126, a documentation module 128, a reference module 130, and a reporting module 132. Referring to FIG. 1, the various modules 126, 128, 130, and 132 operate in conjunction with a database 134, which organizes information related to pharmaceutical preparations. In particular, the database 134 includes a personnel list 136, a fill list 138, an equipment list 140, a set of preparation templates 142, a set of preparation logs 144, a set of evaluation logs 146, a set of reports 148, and reference information 150. The database 134 can be implemented as, for example, a relational database in which information is organized using a set of tables.

Advantageously, the various modules 126, 128, 130, and 132 provide a number of features to facilitate compliance with the set of guidelines for pharmaceutical preparations. For example, the various modules 126, 128, 130, and 132 can provide features to ensure not only that operation and infrastructure of the healthcare organization meet the requirements of USP-NF NF 797 but also that detailed information regarding the operation and the infrastructure is collected so as to allow reporting to the JCAHO. In such manner, the various modules 126, 128, 130, and 132 can provide a number of advantages, such as ensuring ongoing and overall compliance of the healthcare organization with the requirements of USP-NF 797 and ensuring quality and safety of compounded sterile products that are used to treat patients.

Referring to FIG. 1, the security module 126 operates to identify a user of the client computer 102 and to verify that the user is authorized access to features provided by the remaining modules 128, 130, and 132. Verification of the user's identity can be based on, for example, entry of a user name and password combination and comparison of this entry with a corresponding entry in the personnel list 136. It is also contemplated that the user's identity can be verified based on any other suitable technique, such as RFID tagging, biometric scanning, or bar code technology.

As illustrated in FIG. 1, the documentation module 128 operates to track activities related to pharmaceutical preparations. For example, the documentation module 128 can track activities of a preparer in terms of procedures and environments that are used when preparing compounded sterile products. As another example, the documentation module 128 can track activities of a quality assurance pharmacist in terms of evaluation and training that are performed. In the illustrated embodiment, the documentation module 128 collects information related to these activities and then creates or updates the set of preparation logs 144 and the set of evaluation logs 146 based on the collected information.

Referring to FIG. 1, the documentation module 128 provides a set of forms to prompt entry of relevant information. In particular, the documentation module 128 provides a preparation form that is displayed using the web browser 116, and a user of the client computer 102 records preparation activities by “filling out” the preparation form. In a similar manner, the same or a different user records evaluation activities by “filling out” an evaluation form that is displayed using the web browser 116. In some instances, the documentation module 128 provides a form that indicates a set of queries, and a user “fills out” the form by entering responses to the set of queries. For example, the documentation module 128 can provide a preparation form, and the user can be requested to confirm or edit one or more of the following: (a) an identity of a preparer (e.g., a name and a position of the preparer); (b) characteristics of the preparer and other environmental factors (e.g., use of gloves, masks, or other safety equipment); and (c) characteristics of a pharmaceutical product to be prepared (e.g., a drug name, diluents, a risk category, and a preparation procedure). The preparation form can also request that the user specify other relevant information, such as a manufacturer, a lot number, beyond use or expiration date, deviations from approved procedures, a name of a quality assurance pharmacist, a name of an individual who approved or requested the pharmaceutical product, and quality assurance procedures followed.

In the illustrated embodiment, the documentation module 128 generates a preparation form based on selecting a suitable preparation template from the set of preparation templates 142, which can be edited or customized by a pharmacy administrator. Selection of the preparation template can be based on, for example, characteristics of a pharmaceutical product to be prepared or any other suitable criteria. In some instances, the documentation module 128 at least partially “populates” or pre-fills the preparation template with relevant information from the fill list 138, which indicates characteristics of the pharmaceutical product, and the equipment list 140, which indicates safety equipment to be used when preparing the pharmaceutical product. The fill list 138 can be updated by a data feed (e.g., HL-7 messages) from a pharmacy information management system (not illustrated in FIG. 1), thus fulfilling requests for batch preparations, ad hoc preparations, extemporaneous preparations, and emergency or “stat” preparations. In some instances, the documentation module 128 also pre-fills the preparation template with relevant information from the reference information 150, which can be edited or customized by a pharmacy administrator. For example, the preparation template can be pre-filled with an approved procedure for a preparer to review and follow. Selection of the approved procedure can be based on, for example, characteristics of the pharmaceutical product or any other suitable criteria.

Referring to FIG. 1, the reference module 130 operates to maintain and allow access to the reference information 150, which can be viewed by a user of the client computer 102 using the web browser 116. The reference information 150 includes information related to approved procedures, such as Standard Operating Procedures (“SOPs”) for pharmaceutical preparations. The SOPs can be implemented based on, for example, the guidelines for compounded sterile preparations that are set forth in USP-NF 797.

As illustrated in FIG. 1, the reporting module 132 operates to analyze the set of preparation logs 144 and the set of evaluation logs 146 so as to characterize a level of compliance with the set of guidelines for pharmaceutical preparations. In particular, the reporting module 132 generates the set of reports 148 based on evaluating activities that are recorded in the logs 144 and 146. The set of reports 148 can include a variety of reports, such as: (a) reports that indicate compliance or a lack of compliance with SOPs, personnel quality assurance, and product quality assurance; (b) reports that indicate preparation activities by user and by date; (c) reports that indicate a trend or a progression of preparation activities by date, by user, and by pharmaceutical product; (d) reports that indicate results of environment inspections by date, by quality assurance pharmacist, by compounding area, and by safety equipment (e.g., sterility testing results and visual inspection results of garbing and sterile techniques); (e) evaluation reports that indicate training status by date, by preparer, and by competency; (f) activity reports that alert a user regarding time-sensitive activities to be completed within a specific timeframe; and (g) reports that indicate remedial activity related to incompletion of SOPs, failed sterility tests, and failed demonstrations of a preparer's familiarity with approved procedures. It is also contemplated that the set of reports 148 can include a variety of ad hoc or customized reports, such as those generated based on selecting one or more of the following parameters: user; pharmaceutical product; date; activity; inspection type; equipment type; and training type.

The foregoing provides a general overview of the illustrated embodiment. Advantages and features of the illustrated embodiment can be further appreciated with reference to an example of an operational scenario.

First, using the documentation module 128, a preparer accesses a preparation form that is automatically pre-filled with relevant information, such as related to a patient to be treated, a pharmaceutical product to be prepared, a dosage level, a frequency of administration, and a route of administration. This information is specified, either via a Computerized Physician Order Entry (“CPOE”) or through manual entry, in accordance with a physician's order for the patient. It is also contemplated that the preparation form can be blank rather than pre-filled. In this example, the preparer accesses the preparation form using a touch screen and a disposable stylus within a compounding area. Use of the touch screen and the disposal stylus is desirable so as to maintain ongoing sterility of the compounding area. It is also contemplated that access to the preparation form can occur outside of the compounding area, such as in an ante room, using a video monitor and a keyboard or a mouse.

Second, the preparer prepares the pharmaceutical product using an approved compounding procedure. If desired, the preparer can consult the approved compounding procedure using the reference module 130. In connection with preparing the pharmaceutical product, the preparer confirms or edits information indicated in the preparation form, which information is then recorded in the set of preparation logs 144 using the documentation module 128.

Third, a quality assurance pharmacist evaluates the preparer, such as in terms of accuracy and appropriateness of techniques that are used, and, using the documentation module 128, the quality assurance pharmacist enters information that is recorded in the set of evaluation logs 146. In particular, the quality assurance pharmacist enters information regarding competency of the preparer, such as whether remedial training is advisable. Competency of the preparer can be determined based on one or more of the following:
(a) Visual observation: The preparer is observed for use of protective devices, preparation automation devices, protective hoods or barriers, disinfectants, transfer needles, sterile containers, labels, and other safety equipment specified in the SOPs. The observations are recorded in the set of evaluation logs 146.
(b) Training: The preparer is tested with respect to a variety SOPs and using a variety of media. The results of the tests are recorded in the set of evaluation logs 146.
(c) Pyrogen testing: The preparer is requested to prepare a sample pharmaceutical product. The pharmaceutical product, the preparer, and equipment used are sampled and incubated using an agar medium. After an appropriate incubation time, the agar medium is inspected for microbial growth. The results are recorded in the set of evaluation logs 146.

Fourth, using the reporting module 132, a pharmacy administrator generates a variety of reports indicating alerts, timelines, remedial activities, summaries on compounding activities, and details on compounding activities. In addition, the reporting module 132 is used to generate on-line, real-time reports that are made available to JCAHO inspectors when surveying for compliance with USP-NF 797. In such manner, the JCAHO inspectors can be provided with relevant information quickly and on demand. Ad hoc or customized reports can also be generated using a variety of sorting and exporting options. For example, sorting can be performed with respect to a variety of database fields, such as by day, by shift, by preparer, by quality assurance pharmacist, by pharmaceutical product, by diluent, by lot number, by expiration date, or by beyond use date.

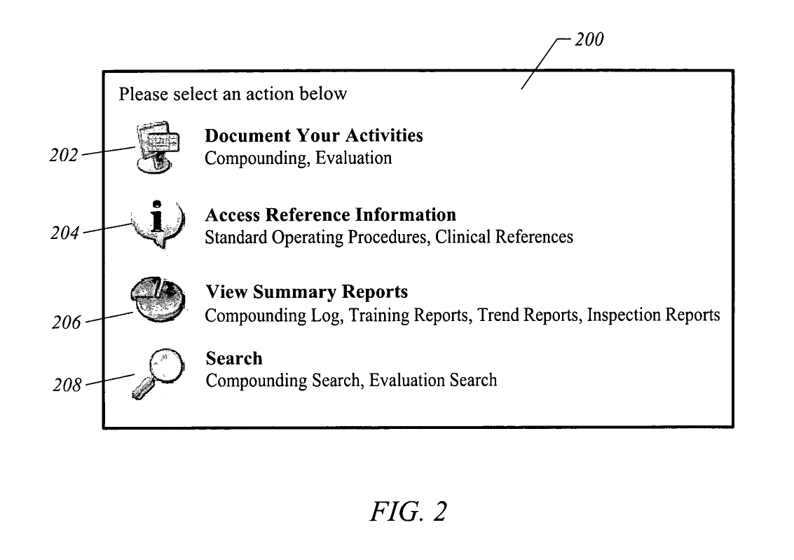

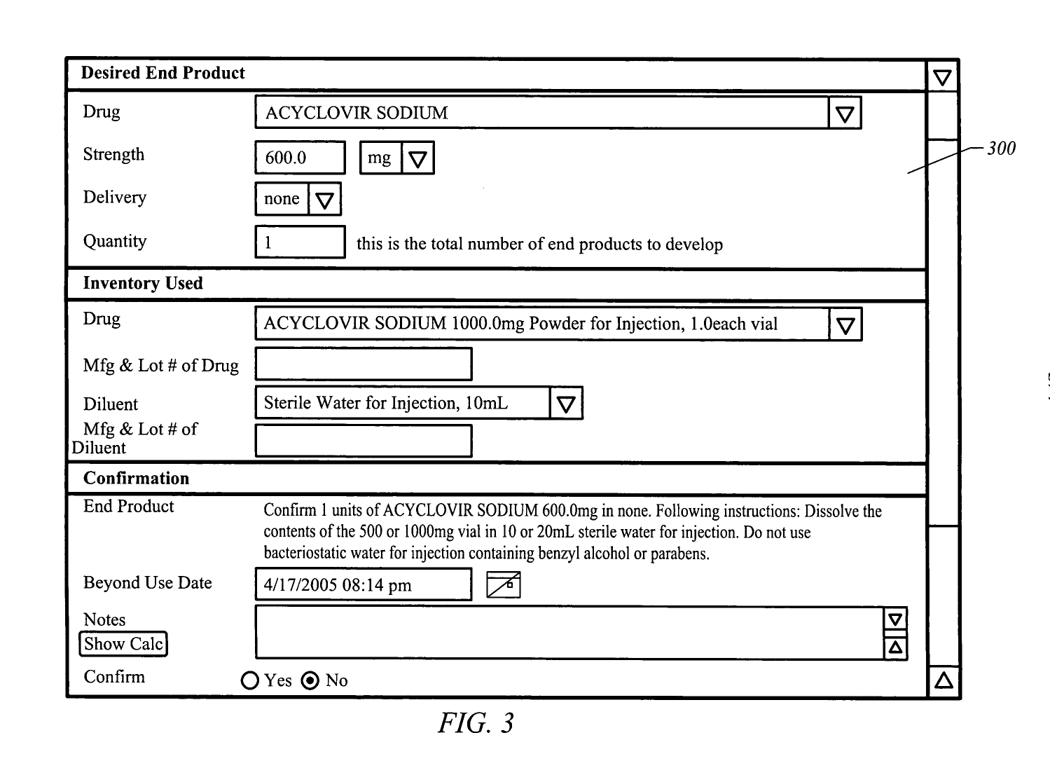

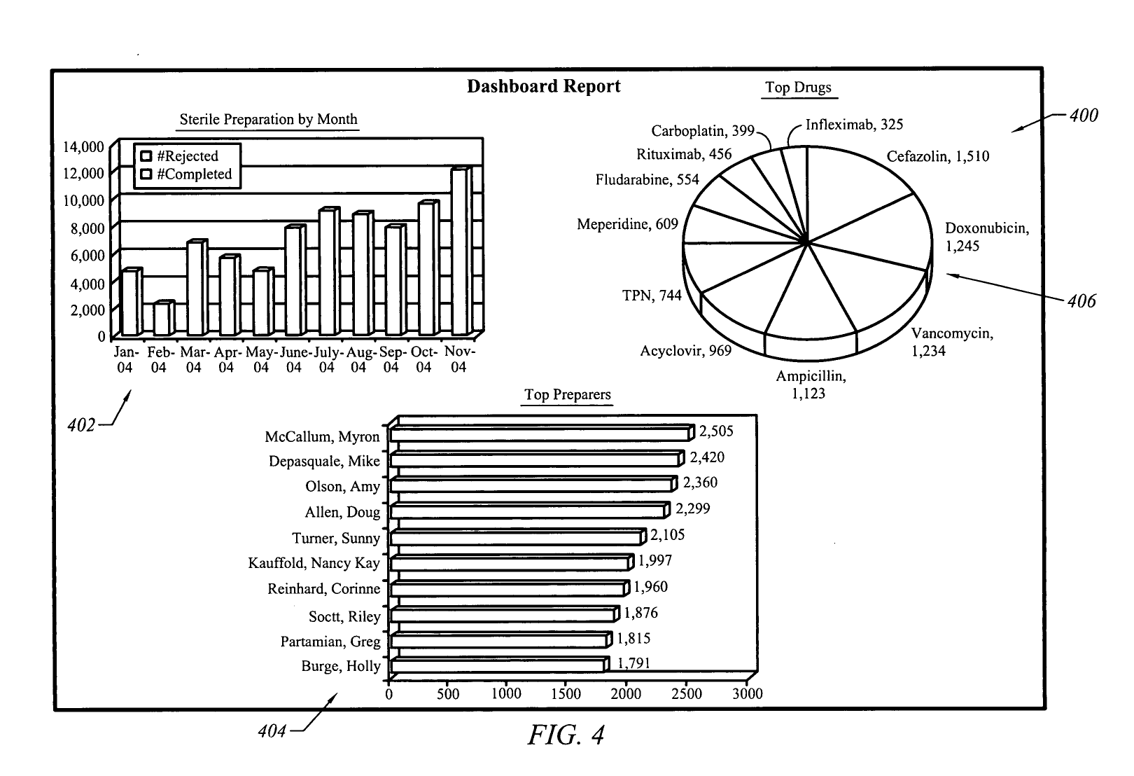

Attention next turns to FIG. 2, FIG. 3, and FIG. 4, which illustrate examples of user-interface screens 200, 300, and 400 that are provided in accordance with an embodiment of the invention.

Referring first to FIG. 2, the user-interface screen 200 displays options 202, 204, 206, and 208, which are used to access features provided by the documentation module 128, the reference module 130, and the reporting module 132. In particular, the option 202 is used to record activities related to compounded sterile preparations, the option 204 is used to view the reference information 150, and the options 206 and 208 are used to create and view summary reports and ad hoc reports characterizing the activities.

As illustrated in FIG. 3, the user-interface screen 300 displays a preparation form that is used to record compounding activities. In the illustrated example, the preparation form is automatically pre-filled with a variety of information regarding a pharmaceutical product to be prepared. The preparation form also indicates an approved compounding procedure for a preparer to review and follow.

Referring next to FIG. 4, the user-interface screen 400 displays a report that characterizes activities related to compounded sterile preparations. In the illustrated example, the report characterizes the activities using a variety of graphical display formats, such as using bar graphs 402 and 404 and a pie chart 406.

At this point, advantages and features of embodiments of the invention can be appreciated. In particular, some embodiments of the invention include features that allow one or more of the following: (a) efficient operation of a compounding area in compliance with standards set forth by the JCAHO; (b) efficient collection, analysis, and reporting of information related to operation of the compounding area; (c) efficient collection, analysis, and reporting of information related to quality assurance procedures performed by a preparer and a quality assurance pharmacist; (d) efficient identification of deficiencies related to operation of the compounding area and efficient development of remedial action to address those deficiencies; (e) efficient delivery of real-time data to JCAHO inspectors in accordance with USP-NF 797; (f) pro-active reporting of alerts to a pharmacy administrator, such as relating to training deadlines and beyond use dates; (g) ensuring ongoing and overall compliance of a healthcare organization with standards set forth by the JCAHO; and (h) ensuring quality and safety of pharmaceutical products and reducing undesirable events resulting from improperly prepared pharmaceutical products.

It should be appreciated that the specific embodiments of the invention described above are provided by way of example, and various other embodiments are encompassed by the invention. For example, while the various modules 126, 128, 130, and 132 and the database 134 are illustrated as residing in the server computer 104, it should be recognized that such configuration is not required in certain implementations. For example, one or more of the various modules 126, 128, 130, and 132 and the database 134 can reside in a separate server computer (not illustrated in FIG. 1) that is connected to the server computer 104. Alternatively, or in conjunction, one or more of the various modules 126, 128, 130, and 132 and the database 134 can reside in the client computer 102. In addition, while not illustrated in FIG. 1, it is contemplated that a database management program can be provided to create the database 134 as well as to facilitate access to the database 134.

Some embodiments of the invention relate to a computer storage product with a computer-readable medium having computer code thereon for performing various computer-implemented operations. The media and computer code may be those specially designed and constructed for the purposes of the invention, or they may be of the kind well known and available to those having skill in the computer software arts. Examples of computer-readable media include, but are not limited to: magnetic storage media such as hard disks, floppy disks, and magnetic tape; optical storage media such as Compact Disc/Digital Video Discs (“CD/DVDs”), Compact Disc-Read Only Memories (“CD-ROMs”), and holographic devices; magneto-optical storage media such as floptical disks; carrier wave signals; and hardware devices that are specially configured to store and execute program code, such as Application-Specific Integrated Circuits (“ASICs”), Programmable Logic Devices (“PLDs”), and ROM and RAM devices. Examples of computer code include, but are not limited to, micro-code or micro-instructions, machine instructions, such as produced by a compiler, and files containing higher-level instructions that are executed by a computer using an interpreter. For example, an embodiment of the invention may be implemented using Java, C++, or other object-oriented programming language and development tools. Additional examples of computer code include, but are not limited to, control signals, encrypted code, and compressed code.

Moreover, an embodiment of the invention can be downloaded as a computer program product, which can be transferred from a remote computer (e.g., a server computer) to a requesting computer (e.g., a client computer or a different server computer) by way of data signals embodied in a carrier wave or other propagation medium via a transmission channel. Accordingly, as used herein, a carrier wave can be regarded as a computer-readable medium.

Some embodiments of the invention can be implemented using computer code in place of, or in combination with, hardwired circuitry. For example, with reference to FIG. 1, various components of the computer system 100 can be implemented using computer code, hardwired circuitry, or a combination thereof.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, process operation or operations, to the objective, spirit and scope of the invention. All such modifications are intended to be within the scope of the claims appended hereto. In particular, while the methods disclosed herein have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A computer-readable medium, comprising:
a documentation module configured to track activities related to pharmaceutical compounding; and
a reporting module configured to evaluate the activities so as to characterize a level of compliance with a pharmaceutical compounding guideline.

2. The computer-readable medium of claim 1, wherein the activities are related to compounded sterile preparations, and the pharmaceutical compounding guideline corresponds to a guideline for the compounded sterile preparations.

3. The computer-readable medium of claim 1, wherein the documentation module is configured to track the activities by collecting information related to the activities and updating a set of preparation logs based on the information.

4. The computer-readable medium of claim 3, wherein the documentation module is configured to collect the information by providing a preparation form that indicates a set of queries.

5. The computer-readable medium of claim 4, wherein the documentation module is configured to generate the preparation form based on a preparation template and a set of characteristics of a pharmaceutical product to be prepared.

6. The computer-readable medium of claim 3, wherein the reporting module is configured to evaluate the activities by analyzing the set of preparation logs.

7. The computer-readable medium of claim 1, wherein the reporting module is configured to generate a report that characterizes the level of compliance with the pharmaceutical compounding guideline.

8. The computer-readable medium of claim 1, wherein the reporting module is configured to generate an alert based on a lack of compliance with the pharmaceutical compounding guideline.
